(19) United States
(12) Patent Application Publication
(10) Pub. No.: US 2004/0171635 A1
(43) Pub. Date: Sep. 2, 2004

(54) Novel tropane esters and methods for producing and using them

(76) Inventors: Nicholas J. Archer, Mid-Lothian (GB); Anita H. Lewin, Chapel Hill, NC (US)

Correspondence Address: FISH & NEAVE, 1251 AVENUE OF THE AMERICAS, 50TH FLOOR, NEW YORK, NY, 10020-1105, US

(21) Appl. No.: 10/729,542

(22) Filed: Dec. 5, 2003

Related U.S. Application Data
(60) Provisional application No. 60/431,609, filed on Dec. 5, 2002.

(51) Int. Cl. C07D 451/04; A61K 31/46

(52) U.S. Cl. 514/304; 546/132

(57) ABSTRACT

This invention relates to novel primary diol tropane esters and related compounds, including methods for making and using those compounds. The compounds of this invention are those of formula (I), (II) or (III):

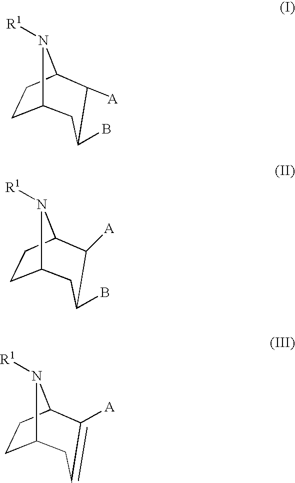

wherein A, B and R¹ are as defined herein. These compounds may be used as therapeutic and prophylactic agents against diseases such as immunoregulatory disorders, neuromuscular disorders, joint disorders, connective tissue disorders, circulatory disorders and pain.

### CROSS REFERENCE TO RELATED APPLICATIONS

This application claims benefit under 35 U.S.C. § 119(e) of U.S. Provisional Application No. 60/431,609, filed Dec. 5, 2002, the disclosure of which is incorporated herein by reference.

### TECHNICAL FIELD

This invention relates to novel primary diol tropane esters and related compounds, including methods for making and using those compounds. The compounds of this invention may be used as therapeutic and prophylactic agents against diseases such as immunoregulatory disorders, neuromuscular disorders, joint disorders, connective tissue disorders, circulatory disorders and pain.

### BACKGROUND

Tropanes are a class of structurally related compounds having diverse biological activities. The class includes benzoylecgonine, ecgonine and ecgonidine, which are known metabolites of cocaine (see, for example, S. M. Roberts et al., “An Assay for Cocaethylene and Other Cocaine Metabolites in Liver Using High-Performance Liquid Chromatography”, Anal. Biochem., 202, pp. 256-61 (1992); D. T. Chia and J. A. Gere, “Rapid Drug Screening Using Toxi-Lab Extraction Followed by Capillary Gas Chromatography/Mass Spectroscopy”, Clin. Biochem., 20, pp.303-06 (1987)). General synthetic routes have also been reported for the preparation of these compounds (see, for example, A. H. Lewin et al., “2β-Substituted Analogues of Cocaine. Synthesis and Binding to the Cocaine Receptor”, J. Med. Chem., 35, pp. 135-40 (1992); M. R. Bell and S. Archer, “L(+)-2-Tropinone”, J. Amer. Chem. Soc., 82, pp. 4642-44 (1960)). This class also includes pseudococaine (the 2-α-carbomethyoxy epimer of cocaine) and its respective analogs.

Benzoylecgonine, ecgonine and ecgonidine and certain derivatives thereof have been reported to possess useful biological activity. Benzoylecgonine, ecgonine and ecgonidine were reported to be useful for the treatment of rheumatoid arthritis and osteoarthritis (see, for example, U.S. Pat. Nos. 4,469,700, 4,512,996 and 4,556,663). Certain covalently-coupled benzoylecgonine, ecgonine and ecgonidine derivatives have been reported to have novel therapeutic features and improved therapeutic properties (see, for example, U.S. Pat. Nos. 5,525,613, 5,763,456, and 6,077,848). 2-β-derivatized analogs have been shown to yield an enhanced rate of absorption into the blood stream, improved solubility and other useful properties have also reported (see, for example, U.S. Pat. Nos. 5,376,667, 5,559,123, 5,663,345). Specific 2-β-derivaitzed analogs include the 1,2-propanediol esters of ecgonine, benzoylecgonine, and ecgonidine.

The 1,2-propanediol esters of benzoylecgonine, ecgonine and ecgonidine are present in Esterom® solution, a topical pharmaceutical product being investigated for the treatment of soft tissue injuries. The most active species in Esterom® solution are believed to be the hydroxypropyl esters of benzoylecgonine that result from the transesterification of benzoylmethylecgonine in 1,2-propanediol. The starting material for Esterom® (benzoylmethylecgonine) is natural R-cocaine that produces two primary diastereoisomeric esters (RR and RS) and two secondary diastereoisomeric esters (RR and RS) on trans-esterification with racemic 1,2 propanediol. The presence of four potentially active molecular species in Esterom® solution has presented difficulties in purification, quantitation, identification of potential degradation products, and in the association of possible toxicological effects and individual biologic/therapeutic effects with the specific mixture and/or the individual molecular species. In addition, the 1,2-propanediol esters of benzoylecgonine, ecgonine and ecgonidine are somewhat susceptible to chemical degradation (for example, hydrolysis, saponification and transesterification).

In view of the above, there remains a need for hydroxypropyl tropane esters and related compounds that have improved physical properties and other advantages over the 1,2-propanediol tropane esters, including (without limitation) a simpler molecular composition, easier purification and characterization, easier assay development, improved stability and/or increased formulation options.

### SUMMARY

The invention described herein fulfills the need described above. In one embodiment, this invention provides a compound of formula (I), (II) or (III):

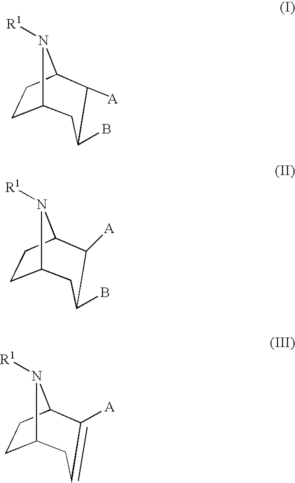

wherein A is —CO—O—CR²—(CR³)ₙ—X;

B is selected from the group consisting of —O—CO—R⁴ and —O—R⁵;

R¹ is selected from the group consisting of H, aryl, arylalkyl, branched or unbranched alkyl, alkenyl and alkynyl, —CO-alkyl, —CO-aryl, and —CO-arylalkyl;

R² is selected from the group consisting of H and branched or unbranched alkyl, alkenyl and alkynyl;

each R³ may be the same or different and is independently selected from the group consisting of H and branched or unbranched alkyl, alkenyl and alkynyl;

R⁴ is selected from the group consisting of H, branched or unbranched alkyl, alkenyl and alkynyl, aryl and arylalkyl;

R⁵ is selected from the group consisting of H, branched or unbranched alkyl, alkenyl and alkynyl, aryl and arylalkyl;

X is selected from the group consisting of OH, SH, amino and halogen;

n is an integer selected from 0, 1, 2, 3, 4, 5 and 6;

and pharmaceutically acceptable esters and salts thereof.

In another embodiment, this invention provides novel methods for producing the compounds of formula (I), (II) and (III).

In yet another embodiment, this invention provides pharmaceutical compositions comprising a compound of formula (I), (II) or (III) and a pharmaceutically acceptable carrier or adjuvant.

In a further embodiment, this invention provides methods for treating, preventing or alleviating the symptoms of immunoregulatory disorders, neuromuscular disorders, joint disorders, connective tissue disorders, circulatory disorders or pain, comprising the step of administering to a mammal, including a human, a pharmaceutically effective amount of a pharmaceutical composition of this invention.

The details of one or more embodiments of the invention are set forth in the description below. Other features, objects, and advantages of the invention will be apparent from the description and claims that follow.

### DETAILED DESCRIPTION

As used herein:

The term “alkyl” (whether used alone or in combination with other terms) refers to a saturated straight chain or branched chain, primary, secondary, or tertiary hydrocarbon radical. In one embodiment of this invention, the alkyl is a C₁-C₁₈ alkyl radical, in another embodiment a C₁-C₁₀ alkyl radical, and in yet another embodiment a C₁-C₆ alkyl radical, including, without limitation, methyl, ethyl, propyl, butyl, pentyl, hexyl, isopropyl, isobutyl, sec-butyl, t-butyl, isopentyl, amyl, and t-pentyl. For the purposes of this invention, any carbon in the alkyl segment may be substituted with oxygen (O), sulfur (S), or nitrogen (N). Further, alkyl moieties useful in the compounds of this invention may optionally be substituted with one or more conventionally used alkyl substituents, such as amino, alkylamino, alkoxy, alkylthio, oxo, halo, acyl, nitro, hydroxyl, cyano, aryl, alkylaryl, aryloxy, arylthio, arylamino, carbocyclyl, carbocyclyloxy, carbocyclylthio, carbocyclylamino, heterocyclyl, heterocyclyloxy, heterocyclylamino, heterocyclylthio, and the like. Unsubstituted alkyls are included as an embodiment of this invention. Propyl is included in another embodiment of this invention.

The term “alkylamino” means an amino segment substituted with one or two alkyl groups (i.e., includes dialkyl amino radicals) wherein the alkyl groups may be the same or different.

The term “alkylaryl” means an aryl radical substituted with one or more alkyl substituents.

The term “alkenyl” means an alkyl radical having one or more double bonds. Alkenyl groups containing three or more carbon atoms may be straight or branched.

The term “alkynyl” means an alkyl radical having one or more triple bonds. Alkynyl groups containing three or more carbon atoms may be straight or branched.

The term “amino” means a —NH₂, —NHR₆, or —NR₆R₇, wherein R₆ and R₇ may be the same or different and represent a conventionally used amino substitutent. In one specific embodiment, R₆ and R₇ are independently selected from the group consisting of optionally substituted alkyl (e.g., lower alkyl), aryl, and alkylarylalkyl.

The term “alkanediol” refers to an alkyl moiety comprising two hydroxyl groups located at any position on the alkyl chain. In one embodiment, the alkanediol is 1,2-propanediol. It should be noted that in some cases, more than two hydroxyl groups may be present on the alkyl chain.

The term “aryl” means a 5-8 membered monocyclic aromatic ring or a polycyclic aromatic ring or ring system having 5-8 ring members in each ring thereof, which may be carbocyclic or heterocyclic and may be unsubstituted or substituted with one or more substituents selected from (but not limited to) alkyl (e.g., lower alkyl), hydroxy, alkoxy (e.g., lower alkoxy), alkylthio, cyano, halo, amino, and nitro. Such aryl radicals may be linked to the remaining portion of the molecule through any position on the ring or substituents that results in a stable compound having the desired activity. Examples of specific aryl groups are phenyl, methylphenyl, dimethylphenyl, aminophenyl, nitrophenyl, hydroxyphenyl, pyrrolyl, thiazolyl, oxazolyl, pyridyl, pyrimidinyl and the like.

The term “arylalkyl” means an alkyl radical substituted with one or more aryl substituents. Examples of specific arylalkyl segments include benzyl, methylbenzyl, dimethylbenzyl, aminobenzyl, nitrobenzyl, hydroxybenzyl, and the like.

The term “benzoylmethylecgonine” or “BME” refers to the chemical entity 3-benzoyloxy-2-carbomethyoxy-8-methyl-8-azabicyclo[3.2.1]octane. BME can exist in four diastereomeric forms (cocaine, pseudococaine, allococaine and allopseudococaine) and each diastereomer has two optical antipodes. Any one of these compounds or any combination of more than one of these compounds is encompassed by the invention herein. BME is typically prepared as a salt (e.g., cocaine HCl) or a base (e.g., cocaine alkaloid) according to known methods.

The term “carbocyclyl” means a segment comprising one or more rings, which may be independently saturated, unsaturated, or aromatic and which contain only carbon ring members. “Carbocyclyl” includes moieties that are unsubstituted or substituted with one or more substituents, which may be selected from (but not limited to) alkyl (e.g., lower alkyl), hydroxy, alkoxy (e.g., lower alkoxy), alkylthio, cyano, halo, amino, and nitro. Suitable carbocycles for use in the compounds of this invention include (without limitation) phenyl, benzyl, indanyl, indenyl, naphthyl, tetralyl, decalyl, cyclopentyl, cyclopentenyl, cyclohexyl, cyclohexenyl and cycloheptyl. Specific carbocycles include (without limitation) cycloalkyl, cycloalkenyl and mono- or bicyclic carbocyclic aromatic rings or ring systems containing from three to ten carbon atoms.

The term “CDI” refers to 1,1′-carbonyldiimidazole.

The term “DCC” refers to dicyclohexylcarbodiimide.

The term “DCU” refers to dicyclohexylurea.

The term “DMAP” refers to 4-dimethylamino pyridine

The term “effective amount” of a compound or a composition according to this invention means an amount which, when administered to a mammal, including a human, in need thereof, produces the desire biological activity.

“Halo” means a halogen radical, i.e., fluoro, chloro, bromo, or iodo.

“Heterocyclyl” means a heterocyclic radical containing one or more rings which may be saturated, unsaturated, or aromatic wherein at least one ring of the radical optionally contains one or more heteroatoms selected from nitrogen (N), oxygen (O), and sulfur (S) in one or more rings. Suitable heterocyclyl for use in the compounds of this invention include radicals of (without limitation) furan, dioxolane, thiophene, pyrrole, pyrazole, triazole, imidazole, pyrrolidine, pyran, pyridine, pyrimidine, morpholine, piperidine, piperazine, oxazole, isoxazole, oxazoline, oxazolidine, oxathiazole, thiazole, isothiazole, thiadiazole, tetrazole, benzofuran, indole, isoindole, quinazoline, quinoline, isoquinoline, purine, pyrrolopyrimidine, pyrazolopyrimidine, pteridine, ketal. In addition, heterocyclyl radicals may contain one or more substituents (i.e., a ring substituent, such as a halogen atom, an alkyl radical, or aryl radical) attached to a ring member atom of the heterocyclyl radical. All stable isomers of heterocyclyl groups are contemplated in this definition.

The terms “2-hydroxypropyl ester”, “2-hydroxypropyl ester derivatives”, “2-HP derivatives” and other similar terms used herein, refer to the 2-hydroxypropyl ester derivatives of tropane acids such as benzoylecgonine, ecgonine and/or ecgonidine. When these terms are used in general herein, they are meant to refer to any of these 2-hydroxypropyl ester derivatives.

The term “lower” means the group to which it is applied preferably has 1-6, and more preferably 1-4, carbon atoms, except in the case of rings (such as cycloalkyl), in which case “lower” signifies 3-6 ring members. Unless otherwise noted to the contrary, all substituents herein to which the term “lower” is applicable (whether that term is actually used or not), shall be preferred as such.

The term “primary diol” means a moiety having two hydroxy groups, each located on a terminal atom of the moiety. Specific primary diols of this invention are branched or unbranched alkyl diols (which may or may not have additional substituents on the carbon atoms of the alkyl chain), including in particular symmetrical primary alkyl diols. 1,3-propane diol is of particular relevance for this invention.

The term “protecting group” means a chemical group that is known in the art to protect an otherwise reactive segment against undesirable reaction during one or more particular synthetic procedures and that is selectively removable under a given set of reaction conditions. Protecting groups may be suitable for use, for example, where a compound of the invention or a synthetic intermediate thereof contains a free amino or carboxylic acid functionality. Suitable protecting groups for such use are well known to those of ordinary skill in the art and include, without limitation, trimethylsilyl, dimethylhexylsilyl, t-butyldimethylsilyl, t-butyldiphenylsilyl,trityl, alkyl groups, acyl groups (such as acetyl and propionyl), methanesulfonyl, and p-toluenesulfonyl. Protecting groups that are especially useful for protecting amide functionalities include (without limitation): aralkoxymethyl (e.g., benzyloxymethyl and substituted benzyloxymethyl); alkoxymethyl (e.g., methoxymethyl and trimethylsilylethoxymethyl); trialkyl/arylsilyl (e.g., trimethylsilyl, t-butyldimethylsily, t-butyldiphenylsilyl); tri alkyl/arylsilyloxymethyl (e.g., t-butyldimethylsilyloxymethyl, t-butyldiphenylsilyloxymethyl); 4-alkoxyphenyl (e.g., 4-methoxyphenyl); 2,4-di(alkoxy)phenyl (e.g., 2,4-dimethoxyphenyl); 4-alkoxybenzyl (e.g., 4-methoxybenzyl); 2,4-di(alkoxy)benzyl (e.g., 2,4-di(methoxy)benzyl); alk-1-enyl (e.g., allyl, but-1-enyl and substituted vinyl e.g., 2-phenylvinyl); allyloxycarbonyl; and lower alkoxycarbonyl and benzyloxycarbonyl. Examples of suitable protecting groups for carboxyl groups are the residue of an ester-forming aliphatic or araliphatic alcohol or of an ester-forming silanol (the alcohol or silanol preferably containing from 1-20 and, more preferably, from 1-10 carbon atoms). Protecting groups that are especially useful for protecting amino functionalities include, without limitation: acyl groups, including acetyl, trifluoroacetyl, benzoyl; and acyloxy groups, including t-butyloxycarbonyl, benzyloxycarbonyl, fluoroethenylmethoxycarbonyl, and the like. Protecting groups may be removed by standard methods after the contemplated reaction has been completed. For a more complete description of protecting groups and their use see T. W. Greene and P. G. M. Wuts, Protective Groups in Organic Synthesis, 2nd ed., John Wiley & Sons, New York, 1991.

The term “substantially all”, when referring to the reactions of this invention, means that more than approximately 80% of the tropane starting material has reacted. In one embodiment, more than approximately 85%, and in another embodiment, more than approximately 90% and in yet another embodiment, more than approximately 95% of the tropane starting material has reacted. The progress of such reactions may be monitored by thin layer chromatography (TLC), high pressure liquid chromatography (HPLC) and other means known to those of ordinary skill in the art.

The term “tropane” refers to a compound having a tropane ring, including without limitation benzoylecgonine, ecgonidine and ecgonine. The term “tropane” includes all isomers of cocaine.

In one embodiment, this invention provides compounds of the following formulae:

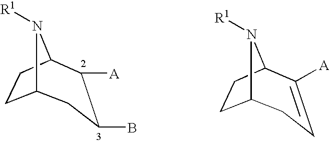

wherein A and B are independently in the α- or β configuration; and

wherein A is —CO—O—CR²—(CR³)ₙ—X;

B is selected from the group consisting of —O—CO—R⁴ and —O—R⁵;

R¹ is selected from the group consisting of H, aryl, arylalkyl, branched or unbranched alkyl, alkenyl and alkynyl, —CO-alkyl, —CO-aryl, and —CO-arylalkyl;

R² is selected from the group consisting of H and branched or unbranched alkyl, alkenyl and alkynyl;

each R³ may be the same or different and is independently selected from the group consisting of H and branched or unbranched alkyl, alkenyl and alkynyl

R⁴ is selected from the group consisting of H, branched or unbranched alkyl, alkenyl and alkynyl, aryl and arylalkyl;

R⁵ is selected from the group consisting of H, branched or unbranched alkyl, alkenyl and alkynyl, aryl and arylalkyl;

X is selected from the group consisting of OH, SH, amino and halogen;

n is an integer selected from 0, 1, 2, 3, 4, 5 and 6;

and pharmaceutically acceptable esters and salts thereof.

In one embodiment, this invention provides compounds of formula (I), (II) and (III):

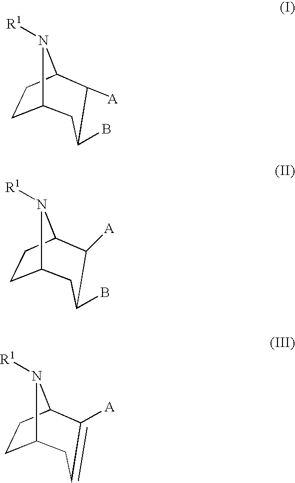

wherein A is —CO—O—CR²—(CR³)ₙ—X;

B is selected from the group consisting of —O—CO—R⁴ and —O—R⁵;

R¹ is selected from the group consisting of H, aryl, arylalkyl, branched or unbranched alkyl, alkenyl and alkynyl, —CO-alkyl, —CO-aryl, and —CO-arylalkyl;

R² is selected from the group consisting of H and branched or unbranched alkyl, alkenyl and alkynyl;

each R³ may be the same or different and is independently selected from the group consisting of H and branched or unbranched alkyl, alkenyl and alkynyl

R⁴ is selected from the group consisting of H, branched or unbranched alkyl, alkenyl and alkynyl, aryl and arylalkyl;

R⁵ is selected from the group consisting of H, branched or unbranched alkyl, alkenyl and alkynyl, aryl and arylalkyl;

X is selected from the group consisting of OH, SH, amino and halogen;

n is an integer selected from 0, 1, 2, 3, 4, 5 and 6;

and pharmaceutically acceptable esters and salts thereof.

In another embodiment, R¹ is selected from the group consisting of H, lower alkyl, —CO-lower alkyl, —CO-phenyl and —CO-benzyl; R² and each R³ may be the same or different and are independently selected from the group consisting of H and lower alkyl; R⁴ is selected from the group consisting of H, lower alkyl, phenyl and benzyl; X is OH and n is an integer selected from 0, 1, 2, 3 and 4.

In yet another embodiment, R¹ is selected from the group consisting of H and —CO-phenyl; R² and each R³ are the same or different and are selected from the group consisting of H and lower alkyl; R⁴ is selected from the group consisting of H, lower alkyl, and phenyl; X is OH and n is an integer selected from 0, 1, 2 and 3.

In a further embodiment, R¹ is selected from the group consisting of H and —CO-phenyl; R₂ and each R³ are the same and are selected from the group consisting of H and lower alkyl; R⁴ is selected from the group consisting of H, lower alkyl, and phenyl; X is OH and n is 2.

In a yet a further embodiment, R¹ is selected from the group consisting of H and —CO-phenyl; R² and each R³ are H; R⁴ is selected from the group consisting of H and phenyl; X is OH and n is 2.

In another embodiment of this invention, when X is OH, the segment —CR²—(CR³)ₙ—X of formula (I), (II) or (III) (part of A) can be a symmetrical primary alkane diol (for example 1,3-propanediol). Symmetry in this segment provides particular synthetic and biological advantages, as described further herein.

This invention also provides a novel method for preparing the primary diol tropane esters of this invention, comprising the steps of:

(a) contacting an appropriately substituted tropane and 1,1′-carbonyldiimidazole to produce an activated tropane ester;

(b) contacting the activated tropane ester with an excess of primary diol to form a reaction mixture; and

(c) maintaining the reaction mixture at a temperature and for a sufficient time for the activated tropane ester to react with the primary diol to form the corresponding primary diol tropane ester.

The method of this invention advantageously produces primary diol tropane esters (such as 1,3-propanediol tropane esters) in good yield and free from impurities that complicate or prevent effective purification of the final product. These compounds may serve as final products themselves, or may be converted to other compounds of this invention. The first steps of the reaction of this invention comprise reacting an appropriately substituted tropane acid and 1,1′-carbonyldiimidazole to form an activated tropane ester, followed by reacting the activated tropane ester with an excess amount of primary diol (such as 1,3-propanediol, also known as 1,3-propylene glycol) to form a reaction mixture. The tropane acid may be added as the free acid or as a salt, such as an acid addition salt (such as a hydrochloride salt). For example, in the case of ecgonine and ecgonidine, their respective hydrochloride salts may be used as the tropane in this reaction. In one embodiment of this invention, the first two steps can advantageously be performed without purification of the activated tropane ester. In a particular embodiment of the invention, the tropane is the free base of benzoylecgonine, ecgonidine, ecgonine, pseudobenzoylecgonine or pseudoecgonine or a salt thereof and the primary diol is 1,3-propanediol. The reaction may be carried out in any suitable organic solvent, including (without limitation) methylene chloride and dimethylformamide (DMF). The reaction may optionally be carried out under an inert gas, such as N₂. Typically, the tropane is contacted with CDI for between 1 minute and 36 hours (after which time, a suspension may be formed and gas evolution may be observed) to form the activated tropane ester of step (a).

The reaction mixture is then formed by contacting the activated tropane ester with an excess amount of the primary diol (such as 1,3-propylene glycol). In particular embodiments of this invention, the excess amount is at least about 6, 10 or 20 equivalents of primary diol to 1 equivalent of tropane. The solution can be stirred or otherwise agitated to promote a steady and efficient reaction.

The reaction mixture should be maintained at a temperature and for a sufficient time for the activated tropane to react with the primary diol and form the corresponding primary diol tropane ester. In one embodiment of this invention, the temperature of the reaction is maintained at between about 0° C. and the boiling point of the solution. For example, the reaction may be run at ambient temperature. The reaction can be monitored to determine when substantially all of the tropane starting material has reacted. The reaction is ordinarily carried out for between about 1 hour and 5 days and in a particular embodiment of this invention, between about 5 hours and 2 days. The amount of tropane starting material remaining in the reaction mixture can be monitored during the course of the reaction using known techniques, such as gas chromatography, high performance liquid chromatography (HPLC), thin layer chromatography (TLC) and/or mass spectrophotometry.

In an additional embodiment of the method of this invention, the primary diol tropane ester can be further isolated or otherwise purified from the reaction mixture. To isolate the final product (for example, after substantially all of the tropane starting material has reacted), the reaction mixture may be filtered (if solid particles have formed) then the final product may be extracted (including by solid phase extraction) or otherwise isolated from the reaction mixture. Depending on the nature of the desired product and other components of the reaction, other means of isolation and purification that may be used include (without limitation) crystallization and chromatography (such as by TLC or HPLC). In the case of final products that are not solids (e.g., oils or gums), it may be convenient to form solid salts that can then be crystallized. In any event, additional purification steps may be employed to further enhance the purity of the final product. Such further purification may involve column chromatography or other suitable techniques known to those of ordinary skill in the art.

Other synthetic methods may also be used to produce compounds of this invention. In the case of primary diol tropane esters, the most common methods include direct esterification of the corresponding acid or conversion of the acid to an acid halide (with reagents such as SOCl₂) followed by esterification. Other esterification methods use coupling agents such as dicyclohexyl carbodiimide (DCC) and dimethylaminopyridine (DMAP). See also Lewin, A. H.; Gao, Y.; Abraham, P.; Boja, J. W.; Khuar, M. J.; Carroll, F. I. J. Med. Chem., 1992, 35(1), 135-140 for methods used for producing tropane esters of simple alcohols.

In certain cases, the 2-α tropane isomers of the compounds of this invention will be preferred (in particular, derivatives and analogs of pseudococaine). Conveniently, pseudococaine may be obtained commercially, but may also be synthesized by treating cocaine with base (for example, by the method described in Calmers, Gossin, CR Hebd Seances Acad Sci 1885, 100, 1143 and Einhorn, Marquardt, Chem Ber. 1890. 23, 472) to obtain pseudoecgonine. Then, the pseudoecgonine is benzoylated with benzoyl chloride (see, for example, Einhom ibid) to yield pseudobenzoylecgonine, which may be esterified with an appropriate primary diol (such as 1,3 propanediol) to yield a compound of this invention (see compounds of formula (I)). Without wishing to be bound by theory, pseudococaine and related 2-α tropanes are believed to bind to voltage gated sodium channels (VGSCs) and tend to be less active in the central nervous system (CNS) than their 2-β counterparts. It will be readily appreciated by those of skill in the art that such reduced CNS activity will be associated with particular therapeutic advantages.

Esters produced from chiral substrates, such as 1,2 propanediol, introduce the possibility of multiple stereoisomers of each regioisomer: for instance, in the case of the ecgonidine, benzoylecgonine and ecgonine esters produced from natural (R)-cocaine, there may be RR and RS primary esters and RR and RS secondary esters. Although such regioisomeric compounds are contemplated in this invention, preferred embodiments include those where the esters are formed with symmetrical substrates (such as 1,3-propanediol). In the case of ecgonidine, benzoylecgonine and ecgonine esters produced from natural (R)-cocaine, only one chiral center exists in the final product.

The compounds of this invention may be used for treating, preventing or alleviating the symptoms of immunoregulatory disorders, neuromuscular disorders, joint disorders, connective tissue disorders, circulatory disorders and pain. As the skilled artisan will appreciate, mixtures of two or more compounds of this invention will also be useful in any application where a single compound of this invention is useful.

While not wishing to be bound by theory, we believe that the compounds of this invention may act as prodrugs. We believe that under physiological conditions, hydrolysis of the C-2 ester group of these compounds slowly occurs, resulting in the formation of the corresponding benzoylecgonine, ecgonine and ecgonidine compounds, respectively. However, the compounds of this invention may also exhibit efficacy in their original, unhydrolyzed form.

The compounds of this invention may be more readily absorbed into the bloodstream than the corresponding benzoylecgonine, ecgonine and ecgonidine compounds because of their increased lipophilicity. We believe that C-2 derivatization increases the lipophilicity of the compounds of this invention, while maintaining or enhancing the desired properties of the corresponding benzoylecgonine, ecgonine and ecgonidine compounds (such as, for example, chelating ability). By administering the compounds of this invention to a patient, greater amounts of the active ingredient will enter the bloodstream and reach the targeted area than if the benzoylecgonine, ecgonine and ecgonidine compounds themselves were administered at the same dosage level. Accordingly, the pharmaceutical effects of the benzoylecgonine, ecgonine and ecgonidine compounds can be enhanced at a lower dosage level without additional side effects.

Furthermore, pharmacological effects which were previously unattainable using particular modes of administration (such as topical administration) can now be realized, due to the decrease in the required dosage level. And because of their increased solubility in solution, the actual administered amount of a pharmaceutical composition containing the compounds of this invention will be decreased, making the composition more easily applied and the treatment regimen more acceptable to the patient. Consequently, it is possible to effectively administer the compounds of this invention in a wide variety of dosage forms.

In addition, certain compounds of this invention may also be able to enter the central nervous system (“CNS”) in an amount effective to treat or prevent certain CNS disorders (such as, for example, Parkinson's disease), without causing adverse side effects commonly associated with conventional centrally-active drugs (e.g., euphoria, tachycardia and vasoconstriction). We believe that in the prodrug form, the C-2 ester may be particularly effective at penetrating the blood/brain barrier but is then hydrolyzed to the corresponding C-2 acid (which could not have passed through the blood/brain barrier). In this manner, pharmaceutically effective amounts of benzoylecgonine, ecgonine and ecgonidine compounds can be successfully targeted at the CNS.

We believe that the compounds of this invention in their native, unhydrolyzed form may also be useful in preventing, treating or alleviating the symptoms of the aforementioned disorders. As the 2-derivatized esters, those compounds may, for example, act peripherally to improve circulation to the afflicted areas. In addition, by increasing the levels of peripherally circulating dopamine (for example, by preventing dopamine re-uptake at the synaptosome), the compounds of this invention may create a chemical sympathectomy.

Although the precise mode of action of the compounds of this invention is not known, one theory is that the compounds of this invention undergo a chelation reaction with the fibers of the muscles and joint capsules, allowing the fibers of the connective tissue to relax and become elongated. This elongation of the connective tissue fibers would result in decreased inflammation by increasing circulation and muscle activity and by improving joint motion. This theory would explain the positive therapeutic results experienced by patients having joint, neuromuscular, connective tissue and circulatory disorders.

Alternatively, the compounds of this invention may act as chelating agents of certain neurotransmitters or co-factors in the body (such as, for example, calcium, sodium and potassium ions). The blood level of free neurotransmitters and co-factors has a direct effect on the functioning of ionic channels and consequently, on intracellular response to various stimuli (such as, for example, intracellular mediation of catecholamine response through the cAMP system). Therefore, the formation of chelation complexes may play a significant role in the pharmacological activity of the compounds of this invention.

Under these chelation theories, the presence of the hydroxy, thiol, amino or halogen moiety at the 2-ε-carbon may be particularly desired, as we believe that chelation occurs at that site. We prefer hydroxy at this position. We also prefer polyols, especially 1,2- or 1,3-diols (i.e., compounds of this invention having a second hydroxy at the zeta- or eta-carbon). Under the chelation theory, these polyols (including the preferred diols), with their multiple chelation sites, will be particularly active.

Another alternative theory involves the intracellular degradation of the compounds of this invention, resulting in the production of certain analgesic, anti-oxidant and anti-inflammatory compounds (such as benzoic acid and salicylic acid). The in vivo production of such pharmaceutically active compounds would procure the benefit of those agents while avoiding many of the side effects associated with their administration (such as gastrointestinal and renal toxicity). The in vivo production of anti-oxidants might explain the impressive immunoregulatory effects shown by the compounds of this invention. Likewise, the production of analgesics and anti-inflammatory agents in the body would also help to explain the mode of action of the compounds of this invention in alleviating pain.

Another possible mode of action involves a reduction in prostaglandin synthesis by inhibiting the action of phospholipase. During conditions of inflammation, pain, fever and platelet aggregation, arachidonic acid is liberated from phospholipid fractions of cell membranes by phospholipase A2. The arachidonic acid is then converted to other products, such as intermediate cyclic endoperoxide prostaglandins. These intermediates produce pain, inflammation and vasoconstriction. Prostaglandins have many other biological actions, including the ability to produce erythema, edema, pain, fever, vasodilation and uterine contractions. Therefore, by inhibiting the synthesis of prostaglandins, many desired physical effects can be realized.

Other possible modes of action include inhibition of chemotaxis of cells implicated in the inflammatory process, inhibition of lysosomal membrane labilization, antagonistic effects on mediators other than prostaglandins (e.g., histamines and bradykinin), inhibition of the biosynthesis of mucopolysaccharides, uncoupling of oxidative phosphorylation, fibrinolytic activity and sulfhydryl-disulfide stabilization.

As can be appreciated by a chemist of ordinary skill in the art, the synthetic schemes described above can be modified to produce any of the compounds of formulas (I), (II) and (III). Such modifications might involve alterations in the starting materials (such as the use of glycols other than propylene glycol or the use of an alcohol in an inert solvent in a transesterification reaction) or the addition of further synthetic steps (such as functional group transformations). Depending on precisely how the synthetic scheme is modified, the specific reaction conditions (such as the precise temperature and reaction times) might also require modification. Since the progress of the reaction can be easily monitored by techniques such as high performance liquid chromatography, gas chromatography, mass spectroscopy, thin layer chromatography, nuclear magnetic resonance spectroscopy and the like, such modifications are well within the skill of the art.

Without wishing to be bound by theory, it is believed that the compounds of this invention have significant advantages over earlier reported diol tropane esters, such as the 1,2-propylene glycol tropane esters. The primary diol tropane esters of this invention are significantly more stable to chemical degradation (including hydrolysis, saponification and transesterification) than their secondary analogs. Furthermore, in the case of compounds of this invention produced from symmetrical primary diols, multiple potentially active molecular species are not formed. Therefore, the challenges associated with difficulties in purification, quantitation and identification of potential degradation products are minimized. In addition, it is less complicated to associate specific toxicological effects and biologic/therapeutic effects with individual molecular species. Compounds and compositions of this invention will have a simpler molecular composition, easier purification and characterization, easier assay development, improved stability and/or increased formulation options. As a result, compounds and compositions of this invention are likely to be easier to develop as pharmaceutically useful products and will have improved activity and fewer toxic side effects as compared to the earlier reported diol tropane esters.

The compounds of this invention may be administered alone or in combination with other compounds, such as, for example, benzoylecgonine, ecgonine and ecgonidine compounds. When compound of formula (I), (II) or (III), or a mixture thereof, is administered together with benzoylecgonine, ecgonine or ecgonidine, the therapeutic efficacy of the latter compounds is enhanced. The pharmaceutical compositions comprising a compound of this invention, or a mixture thereof, may be used in combination with benzoylecgonine, ecgonine and/or ecgonidine. In a specific embodiment such compositions contain at least 5%, or in another embodiment, at least 10%, of the compound or compounds of formulas (I), (II) and (III) (w/w). In a further embodiment, the pharmaceutical compositions of this invention comprise no more than 0.1% cocaine (w/w).

This invention also envisions the administration of the compounds of formulas (I), (II) and (III) in combination with conventional therapeutic agents. Advantageously, such combination therapies utilize lower dosages of those conventional therapeutics, thus avoiding possible toxicity and adverse side effects incurred when those agents are used as monotherapies. For example, the compounds of this invention may be used in combination with conventional cancer drugs (such as, for example, methotrexate, taxol, 5-fluorouracil, cis-platinum, cortisone, nitrogen mustards, thiotepa and nitrosoureas), arthritis drugs (such as, for example, non-steroidal anti-inflammatory agents, penicillamine, methotrexate, cortisone and gold salts) and neurological agents (such as, for example, amantadine, L-DOPA and CNS-anticholinergics).

According to this invention, the compounds of formulas (I), (II) and (III), or mixtures thereof, and the pharmaceutical compositions containing those compounds, may be administered to any mammal, including a human. The compounds and pharmaceutical compositions of this invention may be administered in any pharmaceutically acceptable dosage form, including, but not limited to intravenously, intramuscularly, subcutaneously, intra-articularly, intrasynovially, intrathecally, periostally, intratumorally, peritumorally, intralesionally, perilesionally, by infusion, sublingually, buccally, transdermally, orally, topically or by inhalation. In one embodiment, the administration is topical, transdermal or by inhalation.

Dosage forms may include pharmaceutically acceptable carriers and adjuvants which are known to those of skill in the art. These carriers and adjuvants include, for example, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Adjuvants for topical or gel base forms of the compounds and compositions of this invention include, but are not limited to, sodium carboxymethylcellulose, polyacrylates, waxes, polyoxyethylene-polyoxypropylene-block polymers, polyethylene glycol, propylene glycol and wool fat. For topical applications, 1,3-propylene glycol is a specific adjuvant of interest.

For all administrations, conventionally administered dosage forms may be used. Such forms include, for example, tablet, capsule, caplet, liquid, solution, suspension, emulsion, lozenges, syrup, reconstitutable powder, powder for inhalation, granule, suppository and transdermal patch. Methods for preparing such dosage forms are known (see, for example, H. C. Ansel and N. G. Popovish, Pharmaceutical Dosage Forms and Drug Delivery Systems, 5th edition, Lea and Febiger 1990).

The compounds and pharmaceutical compositions of this invention may be administered in a conventional manner to treat, prevent or alleviate the symptoms of any of the disorders referred to herein and other disorders that benefit from such administration. Such methods and their dosage levels and requirements are well-recognized in the art and may be chosen by those of ordinary skill in the art from the available methods and techniques. Typically, dosage levels range from about 25-200 mg/dose for a 70 kg patient. Although one dose per day is often sufficient, up to 5 doses/day may be given. For oral doses, up to 1500 mg/day may be required. A typical treatment regimen for a 70 kg patient with a joint disorder (such as rheumatoid arthritis) or an immunoregulatory disorder (such as an autoimmune disease) is four doses/day (200 mg/dose), topically applied for two weeks. However, some disorders (such as osteoarthritis) require only 1 dose/day for two days. Once the symptoms of the disorder have receded, maintenance doses can be administered on a p.r.n. basis. As the skilled artisan will appreciate, lower or higher doses than those recited above may be required. Specific dosage and treatment regimens will depend on such factors as the patient's general health status, the severity and course of the patient's disorder or disposition thereto and the judgment of the treating physician.

Immunoregulatory disorders that may be treated with the compounds and compositions of this invention include, but are not limited to: inflammation, autoimmune diseases, allergies (such as, for example, insect bites and stings (e.g., mosquito, fire ant, bee or fly)), poison ivy, poison oak and contact dermatitis.

Neuromuscular disorders that may be treated with the compounds and compositions of this invention include, but are not limited to: amyotrophic lateral sclerosis, multiple sclerosis, skeletal muscle trauma, spasm post-stroke, loss of sensory acuity, weakness, cerebral edema, Reiter's syndrome, polymyositis, Parkinson's disease, Huntington's disease, angina and acute back strain.

Joint disorders that may be treated with the compounds and compositions of this invention include, but are not limited to: frozen shoulder, restricted range of motion, post-fracture contracture, arthritis (such as, for example, rheumatoid arthritis, osteoarthritis, mixed arthritis, psoriatic arthritis, gout, inflammatory gout or juvenile rheumatoid arthritis), bursitis, ankylosing spondylitis, rheumatoid vasculitis and joint rigidity.

Connective tissue disorders that may be treated with the compounds and compositions of this invention include, but are not limited to: systemic lupus, Burger's disease, periarteritis nodosum, proliferative diseases (e.g., keloid scar formation, excessive scar formations, sanctity of scarified fibers and proliferative cancers such as carcinomas and sarcomas), scleroderma and collagen disorders.

Circulatory disorders that may be treated with the compounds and compositions of this invention include, but are not limited to: angina pectoris, myocardial ischemia, gangrene and diabetes (such as diabetes mellitus and diabetes insipidus).

We believe that the compounds and compositions of this invention are especially well suited for use in alleviating pain and alleviating the symptoms of inflammation, Parkinson's disease, acute back strain, restricted range of motion, arthritis, bursitis, ankylosing spondylitis, Burger's disease and myocardial ischemia.

In order that this invention be more fully understood, the following examples are set forth. These examples are for the purpose of illustration only, and are not to be construed as limiting the scope of the invention in any way.

### EXAMPLES

The following specific examples are to be construed as merely illustrative, and not limitative of the disclosure in any way.

Melting points were determined on a Thomas Hoover capillary tube apparatus. Unless otherwise noted, thin layer chromatography was carried out using EM Science or Merck silica gel 60 or RP18 TLC plates; visualization was under UV or in an iodine chamber, as appropriate. Mass spectra were recorded on an Applied Biosystems Sciex API Single Quadrupole Mass Spectrometer using atmospheric pressure chemical ionization. ¹H NMR spectra were obtained on either a Bruker DPX-300 or a Bruker AMX 500 spectrometer. HPLC analysis was carried out using Dynamax Solvent Delivery System Model SD-300, a Rheodyne 7725I injector and a Dynamax Absorbance Detector Model UV-1 or a Sedex Model 75 Evaporative Light Scattering Detector. The ecgonidine, ecgonine and benzoylecgonine acids used as tropane starting material for the methods of this invention can be obtained from a commercial source or alternatively, produced from cocaine by known methods, such as those exemplified herein.

### Example 1

### Production of 1-Hydroxy-3-Propyl Ecgonidine

1.1. Ecgonidine Hydrochloride

A solution of cocaine hydrochloride (15.0 g, 0.044 mol) in conc. HCl (75 mL) was refluxed overnight in a round bottomed flask. After cooling to room temperature the precipitated benzoic acid was removed by filtration and the filtrate was washed with Et₂O (3×25 mL). The aqueous phase was evaporated to a small volume, treated with charcoal and evaporated further. The residue was crystallized from acetone. After a second recrystallization, 6.7 g (65%) of white crystals was collected: m.p. 245-248° C.; [α]_{D}²³−67° (c 1, H₂O).

1.2. 1-Hydroxy-3-Propyl Ecgonidine

A solution of ecgonidine hydrochloride from Example 1.1 (5 g, 25 mmol) and 1,1′-carbonyldiimidazole (CDI) (4 g, 25 mmol) in dry DMF (50 mL) is stirred under N₂. After 10 min a suspension is formed and gas evolution is observed. The reaction mixture is treated with excess 1,3-propanediol (5.5 mL, 75 mmol) and stirring is continued. After approximately 2 days the mixture is filtered and the white solid is washed with CH₂Cl₂. The combined filtrate and washings is concentrated under vacuum and the residual brown oil is dried in vacuo overnight. The oil is partitioned between CH₂Cl₂ (100 mL) and 20% NH₄OH (50 mL). The organic phase is washed twice more with 20% NH₄OH (50 mL), then dried over Na₂SO₄, concentrated and dried in vacuo (3.30 g). This material is purified by column chromatography on SiO₂ (350 g), eluting with CHCl₃:MeOH:NH₄OH (90:10:1).

### Example 2

### Production of 1-Hydroxy-3-Propyl Benzoylecgonine

2.1. Benzoylecgonine

Cocaine hydrochloride (17.0 g, 0.05 mol) was free-based with NH₄OH and extracted into CHCl₃. The combined CHCl₃ layers were dried over Na₂SO₄ and concentrated to afford a white solid. This material was dissolved in H₂O (30 mL) and dioxane (30 mL). The resulting mixture was stirred at 60° C. for seven days. The H₂O/dioxane was removed under reduced pressure yielding 12.5 g (86%) of a white solid: m.p. 198-199° C. {lit (86-92°) 195° C.}; [α]_{D}²²−57° (c 6.1, 100% EtOH) {lit −45° (c 3, 100% EtOH)}.

2.2. 1-Hydroxy-3-Propyl Benzoylecgonine

After stirring at ambient temperature for 24 hours, a solution of anhydrous benzoylecgonine (6.066 g, 21.0 mmol) and 1,1′-carbonyldiimidazole (3.406 g, 21.0 mmol) in CH₂Cl₂ (100 mL) is treated with 1,3-propanediol (10.2 mL, 10.6 g, 138.0 mmol). Stirring is continued as the progress of the reaction is monitored by HPLC. When ester formation slows, the reaction mixture is diluted with CHCl₃ (100 mL) and extracted with 3N HCl (4×40 mL). The combined extract is cooled to 0° C., basified to pH 10 with NH₄OH, and extracted with CHCl₃ (5×40 mL). The combined extract is washed with H₂O, dried with Na₂SO₄, and concentrated. The residue is dried in vacuo overnight to a clear syrup.

2.3. HPLC Analysis

Analysis of the hydroxypropyl benzoylecgonine ester was carried as follows:

Column: Phenomenex Synergi Polar-RP (3*150 mm, 4 μm, 80A)

Solvents: A: 0.1% TFA-H₂O, B: CH₃OH; 30% B; 0.6 mL/min

Detection: 225 nm

2.4. NMR

See section 4.3.

### Example 3

### Production of 1-Hydroxy-3-Propyl Ecgonine

3.1 Ecgonine Hydrochloride

(−)-Cocaine hydrochloride (25 g, 0.07 mol) was dissolved in H₂O (300 mL) in a 2 L three-necked round bottom flask and concentrated HCl (26 mL) was added. After 7 h reflux with stirring, under nitrogen, the reaction mixture was cooled to room temperature and left stirring under nitrogen overnight. The precipitated benzoic acid was removed by filtration and the filtrate was evaporated to a yellow paste. The solid obtained by crystallization from MeOH/Et₂O was washed thoroughly with Et₂O and dried (13.1 g, 0.06 mol, 86%). The m.p. was 246-247° C., {lit 246° C.}; [α]_{D}²³−44.3° (c.1.52, H₂O) {lit-45.2 (0.5%, H₂O)}

3.2. 1 -Hydroxy-3-Propyl Ecgonine

A solution of ecgonine hydrochloride (4.43 g, 0.02 mol) and carbonyldiimidazole (3.24 g, 0.02 mol) in dry DMF (50 mL) is stirred under N₂. After 10 hours a suspension is formed and gas evolution is observed. The reaction mixture is treated with excess 1,3-propanediol (14.7 mL, 0.20 mol) and stirring is continued. After stirring overnight the mixture is concentrated under vacuum and the residual syrup is partitioned between CH₂Cl₂ (100 mL) and 20% NH₄OH (50 mL). The organic phase is washed twice more with 20% NH₄OH (50 mL), then dried over Na₂SO₄, concentrated and dried in vacuo (2.43 g). This material is purified by column chromatography on SiO₂ (325 g), eluting with CHCl₃:MeOH:NH₄OH (90:10:1).

### Example 4

### Production of 1,3-Hydroxylpropyl Benzoylecgonine Ester

4.1. Synthetic method for 1,3-Hydroxylpropyl Benzoylecgonine Ester

To a 25 mL rotary evaporator flask was charged 5.0 g benzoylecgonine (Example 2.1) followed by dichloromethane (100 mL). The contents were concentrated on a rotary evaporator. This procedure was repeated twice with fresh dichloromethane (total 3×100 mL dichloromethane). The contents of the flask were dissolved in 80 mL dichloromethane followed by 2.805 g of 1,1′-carbonyldiimidazole. The clear solution was left stirring under a nitrogen blanket for 22 hours. 1,3-propanediol (8.4 mL) was added and stirring was continued at ambient temperature. After 6 hours, the mixture was diluted with chloroform and extracted with 3N HCl (4×20 mL). The combined acidic extract was cooled to 0-5° C. and basified to pH 9 with ammonia, then extracted into chloroform (5×20 mL). The chloroform extract was washed with water (50 mL) before being dried over MgSO₄ and concentrated on the rotary evaporator (both at 50° C.). The resulting residue was a clear oil (5.1 g, >99% purity by HPLC). C₁₉H₂₅N₁O₅, M=347, M−75=272 (loss of diol side group) and overall MS consistent with the title compound.

4.2. HPLC Analysis

Analysis of 1,3-hydroxylpropyl benzoylecgonine ester was carried as follows:

Column: Phenomenex Synergi Polar-RP (250*4.6 mm, 4 μm, 80A)

Eluent: A: 0.1% TFA-H₂O, B: CH₃OH; 40%

Flow rate: 1 mL/min

Detection: UV@235 nm

The retention time was:

Rf (min): 13.31 min

4.3. NMR

Assignment of the ¹H and ¹³C NMR spectra of benzoylecgonine 1,3-propanediol ester.

| Carbon | Multiplicity | ¹³C Shift | ¹H Shift |
|---|---|---|---|
| 1 | CH | 64.9 | 3.53 |
| 2 | CH | 51.0 | 3.04 |
| 3 | CH | 66.6 | 5.24 |
| 4 | CH₂ | 35.5 | 2.42, 1.89 |
| 5 | CH | 61.5 | 3.28 |
| 6* | CH₂ | 25.7 | 2.13, 1.72 |
| 7* | CH₂ | 25.1 | 2.13, 1.72 |
| 9 | CH₃ | 40.9 | 2.18 |
| 10 | C═O | 171.2 | — |
| 11 | C═O | 166.0 | — |
| 12 | C | 130.2 | — |
| 13 | CH | 128.3 | 7.39 |
| 14 | CH | 129.6 | 7.97 |
| 15 | CH₂ | 133.0 | 7.52 |
| 16 | CH₂ | 62.8 | 4.44, 4.24 |
| 17 | CH₂ | 31.8 | 1.90 |
| 18 | CH₂ | 60.1 | 3.72 |

*may be reversed

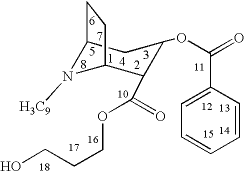

### Example 5

### Production of 3-Hydroxypropyl Benzoylecgonine Ester

5.1. Synthetic Method for 3-Hydroxypropyl Benzoylecgonine

To a solution of thoroughly dried benzoylecgonine (75 g, 0.259 mol) in CH₂Cl₂ (1300 mL) was added CDI (42.03 g, 0.259 mol) under N₂. After stirring overnight at r.t. a solution of 3-hydroxypropanol (distilled) (18.72 mL, 2.59 mol) in dry DMF (140 mL) was added using an oven-dried addition funnel. The homogeneous mixture was stirred overnight at r.t. under N₂. The reaction mixture was then transferred to a 4 L separatory funnel and extracted with chilled 3N HCl (4×200 mL). The aqueous phase was chilled in an ice bath and basified to pH 10 with conc. NH₄OH leading to precipitation of a white solids. The basified aqueous was extracted into CHCl₃ (3×200 mL) and the combined organic extract was washed sequentially with 15% NH₄OH (3×200 mL), H₂O (3×200 mL), and sat. NaCl (200 mL). The extract was then dried over Na₂SO₄ and evaporated to leave 94.02 g of a syrup. Vacuum drying at 45° C. left 85.48 6 (95% of theory).

4.2. HPLC Analysis

Analysis of 3-hydroxypropyl benzoylecgonine ester was carried as follows:

Column: Phenomenex Synergi Hydro RP 150×3

Eluent: 48% CH₃CN; 40% 0.055 M Na₂HPO₄; 12% H₂O pH adjusted to 6.5 with H₃PO₄

Flow rate: 0.5 mL/min

UV: 235 nm

Rt: 2.8 min

Note: An impurity was observed at 10.6 min. Mass spectral analysis gave m/zs 619, consistent with m+1 for 1,3-bis(benzoylecgonine)propane (C₃₅H₄₂N₂O₈). The impurity level was 0.95 mole %.

### Example 6

### Production of 3-Hydroxypropyl Benzoylecgonine Bisulfate

5.1. Synthetic method for 3-Hydroxypropyl Benzoylecgonine Bisulfate

A solution of H₂SO₄ in acetone was prepared by the slow addition of H₂SO₄ (12.9 mL, 0.243 mol) to ice-chilled acetone (54 mL) and this solution was added slowly an ice-chilled solution of 3-hydroxypropyl benzoylecgonine (84.32 g, 0.243 mol) in acetone (407 mL). The solution was warmed to 60° C. and sonicated. After coming to r.t. the solution was placed in the freezer. The precipitated solid was collected by filtration and washed with acetone (99.17 g, 92% yield). HPLC showed 0.95 mole % of 1,3-bis(benzoylecgonine)propane. Recrystallization from aqueous acetone gave 78.74 (79%) with 0.32 mole % of 1,3-bis(benzoylecgonine)propane. Recrystallization of a small portion did not further improve the purity. To remove the acetone the salt was dissolved in absolute ethanol and the solvent was evaporated. The solid was then dried in vacuo at 45° C. M.p. 202° C.; Anal. Calcd for C₁₉H₂₇NO₉S: C, 51.27; H, 6.11; N, 3.14. Found: C, 51.31; H, 6.10, N, 3.12.

All publications, patent applications, patents, and other documents cited herein are incorporated by reference in their entirety. In case of conflict, the present specification, including definitions, will control. In addition, the materials, methods, and examples shown above are illustrative only and not intended to be limiting.

A number of embodiments of the invention have been described. Nevertheless, it will be understood that various modifications may be made without departing from the spirit and scope of the invention. Accordingly, other embodiments are within the scope of the following claims.

What is claimed is:

1. A compound of the following formulae: 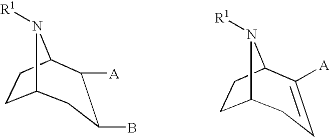
wherein A and B are independently in the α- or β configuration; and
wherein A is —CO—O—CR²—(CR³)ₙ—X;
B is selected from the group consisting of —O—CO—R⁴ and O—R₅;
R¹ is selected from the group consisting of H, aryl, arylalkyl, branched or unbranched alkyl, alkenyl and alkynyl, —CO-alkyl, —CO-aryl, and —CO-arylalkyl;
R² is selected from the group consisting of H and branched or unbranched alkyl, alkenyl and alkynyl;
each R³ may be the same or different and is independently selected from the group consisting of H and branched or unbranched alkyl, alkenyl and alkynyl
R⁴ is selected from the group consisting of H, branched or unbranched alkyl, alkenyl and alkynyl, aryl and arylalkyl;
R⁵ is selected from the group consisting of H, branched or unbranched alkyl, alkenyl and alkynyl, aryl and arylalkyl;
X is selected from the group consisting of OH, SH, amino and halogen;
n is an integer selected from 0, 1, 2, 3, 4, 5 and 6;
and pharmaceutically acceptable esters and salts thereof.

2. A compound of formula (I), (II) or (III): 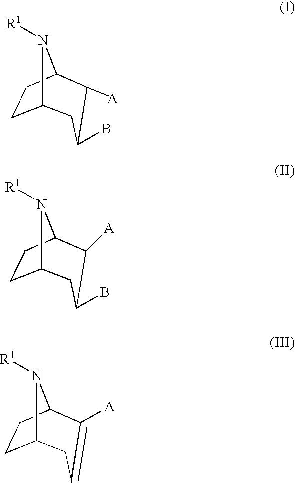
wherein A is —CO—O—CR²—(CR³)ₙ—X;
B is selected from the group consisting of —O—CO—R⁴ and —O—R⁵;
R¹ is selected from the group consisting of H, aryl, arylalkyl, branched or unbranched alkyl, alkenyl and alkynyl, —CO-alkyl, —CO-aryl, and —CO-arylalkyl;
R₂ is selected from the group consisting of H and branched or unbranched alkyl, alkenyl and alkynyl;
each R³ may be the same or different and is independently selected from the group consisting of H and branched or unbranched alkyl, alkenyl and alkynyl
R⁴ is selected from the group consisting of H, branched or unbranched alkyl, alkenyl and alkynyl, aryl and arylalkyl;
R⁵ is selected from the group consisting of H, branched or unbranched alkyl, alkenyl and alkynyl, aryl and arylalkyl;
X is selected from the group consisting of OH, SH, amino and halogen;
n is an integer selected from 0, 1, 2, 3, 4, 5 and 6;
and pharmaceutically acceptable esters and salts thereof.

3. The compound according to claim 2, wherein X is OH.

4. The compound according to claim 3, wherein the segment —O—CR²—(CR³)ₙ—X is a symmetrical primary alkyl diol.

5. The compound according to claim 4, wherein n is 0, 1, 2 or 3.

6. The compound according to claim 5, wherein n is 2.

7. The compound according to claim 6, wherein R² and R³ are H.

8. A method for producing a primary diol tropane ester, comprising the steps of
(a) contacting an appropriately substituted tropane and 1,1′-carbonyldiimidazole to produce an activated tropane ester;
(b) contacting the activated tropane ester with an excess of primary diol to form a reaction mixture; and
(c) maintaining the reaction mixture at a temperature and for a sufficient time for the activated tropane ester to react with the primary diol to form the corresponding primary diol tropane ester.

9. The method according to claim 8, wherein the primary diol is 1,3-propanediol.

10. The method according to claim 9, wherein the tropane is ecgonine, benzoylecgonine or ecgonidine.

11. The method according to claim 10, wherein the reaction of step (b) is carried out in dry DMF.

12. The method according to claim 11, wherein the excess of primary diol is at least about 2 equivalents to 1 equivalent of tropane.

13. The method according to claim 8, wherein the reaction of step (b) is carried out under an inert gas.

14. The method according to claim 13, wherein the inert gas is nitrogen.

15. The method according to claim 8, wherein reaction of step (b) is carried out in methylene chloride.

16. The method according to claim 8, further comprising the step of isolating the primary diol tropane ester from the reaction mixture.

17. The method according to claim 16, wherein the isolation is performed by extraction.

18. The method according to claim 17, further comprising the step of purifying the isolated primary diol tropane ester.

19. The method according to claim 18, wherein the purification is performed by column chromatography.

20. A pharmaceutical composition comprising a compound according to claim 2 and a pharmaceutically acceptable carrier or adjuvant.

21. The pharmaceutical composition according to claim 20, wherein the pharmaceutically acceptable carrier or adjuvant is propylene glycol.

22. The pharmaceutical composition according to claim 21, comprising at least one additional ingredient selected from the group consisting of methotrexate, taxol, 5-fluorouracil, cis-platinum, cortisone, nitrogen mustards, thiotepa and nitrosoureas, non-steroidal anti-inflammatory agents, penicillamine, methotrexate, cortisone and gold salts, amantadine, L-DOPA and CNS-anticholinergics.

23. The pharmaceutical composition according to claim 22, wherein the composition is in an administering dosage form selected from the group consisting of a tablet, capsule, caplet, liquid, solution, suspension, emulsion, lozenges, syrup, reconstitutable powder, granule, suppository and transdermal patch.

24. The pharmaceutical composition according to claim 23, wherein the administering dosage form is a topical solution or a transdermal patch.

25. A method for treating, preventing or alleviating the symptoms of immunoregulatory disorders, neuromuscular disorders, joint disorders, connective tissue disorders, circulatory disorders or pain, comprising the step of administering to a mammal, including a human, a pharmaceutically effective amount of the pharmaceutical composition according to claim 20.

26. The method according to claim 25, wherein the pharmaceutical composition is administered intravenously, intramuscularly, subcutaneously, intra-articularly, intrasynovially, intrathecally, periostally, intratumorally, peritumorally, intralesionally, perilesionally, by infusion, sublingually, buccally, transdermally, orally, topically or by inhalation.

27. The method according to claim 26, wherein the pharmaceutical composition is administered transdermally, topically or by inhalation.

28. The method according to claim 25, wherein the disorder is selected from the group consisting of pain, inflammation, autoimmune diseases, allergies, poison ivy, poison oak, contact dermatitis, amyotrophic lateral sclerosis, multiple sclerosis, skeletal muscle trauma, spasm post-stroke, loss of sensory acuity, weakness, cerebral edema, Reiter's syndrome, polymyositis, Parkinson's disease, Huntington's disease, angina, acute back strain, frozen shoulder, restricted range of motion, post-fracture contracture, arthritis, bursitis, ankylosing spondylitis, rheumatoid vasculitis, joint rigidity, osteoarthritis, mixed arthritis, psoriatic arthritis, gout, inflammatory gout, juvenile rheumatoid arthritis, systemic lupus, Burger's disease, periarteritis nodosum, proliferative diseases, scleroderma, collagen disorders, angina pectoris, myocardial ischemia, gangrene and diabetes.

29. The method according to claim 28, wherein the disorder is pain, inflammation, Parkinson's disease, acute back strain, restricted range of motion, arthritis, bursitis, ankylosing spondylitis, Burger's disease and myocardial ischemia.
